(19) United States
(12) Patent Application Publication
(10) Pub. No.: US 2016/0139051 A1
(43) Pub. Date: May 19, 2016

(54) APPARATUS AND METHOD FOR DISTINGUISHING BETWEEN DIFFERENT TISSUE TYPES USING SPECIFIC RAMAN SPECTRAL REGIONS

(71) Applicants: HENRY FORD HEALTH SYSTEM, Detroit, MI (US); WAYNE STATE UNIVERSITY, Detroit, MI (US)

(72) Inventors: Gregory William AUNER, Livonia, MI (US); Rachel KAST, Holly, MI (US); Steven KALKANIS, Bloomfield, MI (US); Mark ROSENBLUM, West Bloomfield, MI (US)

(21) Appl. No.: 14/904,144

(22) PCT Filed: Jul. 11, 2014
(86) PCT No.: PCT/US2014/046391

§ 371 Date: Jan. 11, 2016

Related U.S. Application Data
(60) Provisional application No. 61/844,926, filed on Jul. 11, 2013.

(51) Int. Cl. G01N 21/65; A61B 5/1455; A61B 5/145; A61B 5/00

(52) U.S. Cl. CPC G01N 21/65; A61B 5/4064; A61B 5/1455; A61B 5/14546; G01N 2201/0221

(57) ABSTRACT

A portable apparatus and method for distinguishing between different tissue types, such as normal tissue, necrotic tissue, and tumor tissue are provided, where the apparatus includes a housing and a plurality of Raman spectrometers disposed within the housing, each spectrometer having a different spectral region. A processor is provided in communication with the plurality of spectrometers, the processor analyzing output from the plurality of spectrometers to identify the tissue type of the tissue sample. A method of selecting the spectral regions which provide a desired combined classification accuracy for determining the tissue type is also provided.

### CROSS-REFERENCE TO RELATED APPLICATIONS

This application claims the benefit of U.S. provisional application Ser. No. 61/844,926 filed Jul. 11, 2013, the disclosure of which is hereby incorporated in its entirety by reference herein.

### TECHNICAL FIELD

Embodiments relate to an apparatus and method for distinguishing between different tissue types, such as brain tissue, using specific Raman spectral regions.

### BACKGROUND

Glioblastoma (GBM) is an extremely aggressive primary brain tumor. Despite multimodality therapy, including maximal surgical resection and adjuvant radiation and chemotherapy, the prognosis for GBM patients remains dismal, with an average life expectancy around 12-18 months. A significant factor in determining patient outcomes is the completeness of resection of the malignant tissue. However, GBM is a diffusely infiltrating glioma, and the tumor margins are difficult to identify intraoperatively, even with the assistance of intraoperative neuronavigation

Establishing a histopathological diagnosis of GBM is essential for initiating therapy. Intra-operative consultations with frozen sections are often performed to help confirm the presence of tissue diagnostic of GBM in the biopsy samples. However, performing frozen sections is limited by the time taken during ongoing neurosurgery, the need for an experienced neuropathologist to interpret the frozen sections, and various processing artifacts that may lead to sub-optimal histological evaluation. In areas of eloquent brain where glioma cells infiltrate normal tissue, patient functionality outcomes may be improved by sparing normal brain while leaving small populations of residual cells, but available tools are often too imprecise for this level of discrimination.

Intra-operative magnetic resonance imaging (iMRI) has been suggested as a potential tool for identifying tumor tissue along the surgical margins to aid in resection. However, uptake of contrast enhancement in areas of diffuse tumor is not as robust as in the tumor core. Furthermore, iMRI-assisted surgery is limited by its significant cost, the time of imaging, and its accessibility confined to major cancer centers.

### SUMMARY

In one embodiment, a portable apparatus for distinguishing between different tissue types in a tissue sample is provided, the apparatus including a housing, and a plurality of Raman spectrometers disposed within the housing, each spectrometer having a different spectral region. A processor is provided in communication with the plurality of spectrometers, the processor analyzing output from the plurality of spectrometers to identify the tissue type of the tissue sample.

In another embodiment, a method for distinguishing between different tissue types in a tissue sample is provided, the method including providing a portable apparatus having a housing, a light source disposed within the housing, and a plurality of Raman spectrometers disposed within the housing, each spectrometer having a different spectral region. The method further includes illuminating the tissue sample using the light source, receiving light from the tissue sample with the plurality of spectrometers, and analyzing output from the plurality of spectrometers to identify the tissue type of the tissue sample.

In another embodiment, a method for distinguishing between different tissue types in a tissue sample using different Raman spectral regions is provided. The method includes (a) selecting a first spectral region which provides a best classification accuracy between the tissue types, (b) selecting a next spectral region that provides a next best classification accuracy between the tissue types, (c) repeating step (b) until a plurality of spectral regions are selected that, when combined, provide a desired combined classification accuracy, and (d) analyzing the tissue sample with the plurality of selected spectral regions to determine the tissue type in the tissue sample.

### BRIEF DESCRIPTION OF THE DRAWINGS

FIG. 1 is a graph of the mean Raman spectrum for regions of normal grey matter, necrosis, and GBM;

FIG. 2 is a graph of discriminant function analysis scores for normal grey matter, necrosis, and GBM tumor tissue;

FIG. 3 is a schematic illustration of a portable apparatus with a plurality of Raman spectrometers with specific spectral regions according to an embodiment;

FIG. 4 is a top perspective view of a portable Raman spectrometer apparatus according to an embodiment; and

FIG. 5 is a bottom perspective view of the portable apparatus of FIG. 4.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Embodiments include a portable Raman spectroscopy apparatus and method for the in vivo identification and distinction between normal tissue, necrotic tissue, and tumor tissue and their boundaries in real time, such as during surgery. Raman spectroscopy is a non-destructive surface technique which provides a molecular signature of the region under examination. When light is incident on a sample, most of the light is scattered back at the same energy and wavelength. However, in rare cases (1 in 10⁷ photons), there is an energy exchange between the incident photon and the molecule under examination causing the scattered photon to shift its wavelength, termed the “Raman effect”.

As is known in the art, Raman spectrometers use focused laser light and highly accurate optical systems to rapidly measure a molecular signature of a region under examination. Raman spectroscopy can be performed at several points within a region of tissue to provide a molecular map of the tissue. Because Raman spectroscopy is non-destructive and is not significantly impacted by water, it is an ideal tool for mapping regions of tumor and necrosis in the brain. Preliminary in vivo studies of brain tissue have been performed using fiber optics connected to full-size (benchtop) Raman spectrometers. However, these spectrometers are large and expensive, and the output spectrum must undergo significant processing to provide a diagnosis.

A typical Raman spectrum provides hundreds, or even thousands, of data points, each representing a specific wavelength or energy shift. Traditional statistical methods are not suited for this type of data. Compression methods such as principal component analysis have been used to reduce data to a few significant variables. However, this ignores the wealth of molecular data present in the Raman spectrum. Frequently, compressed data is then used for clustering methods to identify like regions within areas of tissue. These unsupervised methods are then correlated with histology, and classification methods are developed based on the clusters. While other blind methods, such as support vector machines, have provided high accuracy, these continue to ignore the molecular significance of the Raman spectra.

In an embodiment of the disclosed apparatus and method, a selected group of peaks or regions in a Raman spectrum which provide specific biological (molecular) information, rather than the entire Raman spectrum, may be used to identify GBM tumor tissue, necrosis, and normal brain tissue and their boundaries. A study was performed to distinguish between normal brain (grey matter), necrosis, and GBM regions in banked frozen tissue samples using Raman spectroscopy. Discriminant function analysis was used for spectral identification, to allow for biologically relevant interpretation of the model structure. Homogenous regions of normal grey matter, necrosis, and GBM were identified. Using data from these ‘known’ areas, a select group of Raman peaks was directed into discriminant function analysis for tissue identification. Using discriminant function analysis allows for rapid, accurate identification of neural tissue without loss of meaningful biologic data.

In the study, Raman spectroscopy was performed with an InVia Raman microscope (Renishaw, Gloucestershire, UK), using a 785 nm excitation laser, 1200 l/mm grating, and a 576×400 pixel thermoelectric-cooled charge-coupled device (CCD). A 50× Nikon plan-fluor objective with a numeric aperture of 0.45 and working distance of 4.5 mm was used for measurements, with an approximate spot size of 5×30 μm when focused on the sample. When optimized, laser power at the sample is approximately 115 mW at 100% power, and spectral resolution is 0.7 cm⁻¹. Actual resolution varied from 0.82 to 0.98 cm⁻¹. Prior to daily measurement, system calibration was performed using a silicon control sample. Data was measured over a spectral range of 600-1800 cm⁻¹. Each spectrum consisted of 1 accumulation with an integration time of 10 seconds and a laser power of 50%. At least two distinct regions were measured on each tissue. For each measurement, a region of interest was identified based on the following criteria: a) the region was level to ensure consistent focus throughout the area, and b) the region was of recognizable features or near identifiable orientation markers for easy correlation with the H&E section. Renishaw Wire software then automatically subdivided the region into a 25-μm grid. A Raman measurement was performed at each grid point in the selected measurement region.

Following Raman measurement, each region measured with Raman spectroscopy was identified and photographed at 20× and 40× magnification on the adjacent H&E slide, when possible. Some regions could not be correlated due to folding or stretching in the frozen sections, or lack of orientation markers within the tissue. An experienced neuropathologist examined each H&E slide and marked distinct regions of normal grey matter, tumor, or necrosis. Areas of tumor were further noted as suspicious for tumor, diffuse glioma, and GBM. Freeze artifact was noted when it was present. Images of each map region were reviewed, and the location of each region was compared to the marked regions on the H&E slide to reach a final gold-standard diagnosis for each area studied. For each region measured, the H&E slide and recorded images of each region measured by Raman spectroscopy were reviewed by an experienced neuropathologist.

Spectra were preprocessed using proprietary software by spike elimination, background subtraction, vector normalization, and Whitaker smoothing prior to statistical analysis. Following processing, spectra were individually reviewed to remove spectra containing obvious measurement error, such as missing data, failure of cosmic ray removal, or CCD overload.

Raman data from homogenous regions of normal grey matter, necrosis, and GBM which did not display freeze artifact were identified based on histologic validation. The data was further randomly divided into a model training group and a validation group. Three regions each of normal grey matter, necrosis, and GBM were included in the training group. A third group was established for further validation, which consisted of regions identified as homogenous but displaying freeze artifact. The overall mean of each type of tissue in the training group was calculated and plotted using the Statistical Analysis Software (SAS) procedure PROC MEANS. All distinct Raman peaks and shoulders were identified within the GBM, normal grey matter, and necrosis spectra. These distinct peaks were input to discriminant function analysis using SPSS (i.e., a statistical analysis package from IBM) to develop a tissue identification algorithm. This algorithm was then applied to the validation groups (with and without freeze artifact).

A total of 17,138 Raman spectra were collected from 95 distinct regions of 40 brain tissues. An average of 180 individual spectra were measured from each region (minimum 35, maximum 494, standard deviation 76). The 40 tissues were extracted from 17 donors; 12 with a GBM diagnosis and five with a non-tumor (epilepsy) diagnosis. Among the GBM donors, 58.3% were male ( 7/12), with an average age of diagnosis at 63.9 years (range: 47 to 76 years), with diagnosis occurring between years 1993 and 2000, and an average time of 353.5 days (range: 8 to 806 days). Among the non-tumor donors, 60% were males (⅗), with surgery occurring between 1993 and 1996 at an average age of 31.8 years (range: 19 to 45 years).

Because of the sensitivity of the discriminant function analysis algorithm, regions which contained a mix of cell types (i.e., diffuse glioma, tumor/necrosis border, etc.) were excluded from this study. H&E review showed that a majority of the normal tissue provided for this study was grey matter. Previous studies have shown a distinct biochemical and Raman difference between grey and white matter, and other brain structures, such that the system and method disclosed herein may also be used to distinguish between these tissue types. However, for this study, regions of white matter and leptomeninges were excluded from analysis. Likewise, regions of severe hemorrhage were excluded, as they have been shown to have a unique Raman signature.

Three regions each of normal grey matter, necrosis, and GBM were randomly assigned to a model training set (1396 spectra), while the rest of the data was reserved as the primary validation set (1759 spectra). The mean Raman spectrum was calculated for normal grey matter, necrosis, and GBM in the training data. Raman shoulders and peaks were identified at 927, 934, 954, 958, 977, 1003, 1030, 1061, 1081, 1107, 1122, 1154, 1172, 1206, 1239, 1255, 1259, 1266, 1300, 1313, 1334, 1397, 1419, 1441, 1518, 1552, 1578, 1581, 1604, 1614, 1616, 1657, 1659, and 1735 cm⁻¹. FIG. 1 shows the mean spectrum for each tissue, with peaks and major bands for lipid, protein, cholesterol/cholesterol esters, and nucleic acids labeled. In cases where a Raman peak can correspond to multiple types of molecules, all potential molecules are shown.

There is a general consensus that the Raman spectrum of normal brain tissue is dominated by lipids (1063, 1081, 1127, 1268, 1298, 1313, 1397, 1440, 1657 cm⁻¹) and cholesterol (1440, 1670-1675, 1735 cm⁻¹). Necrosis is characterized by an increased protein content (phenylalanine at 1003, 1032, and 1208 cm⁻¹, tyrosine [peaks below 900 cm⁻¹], tryptophan at 1340 cm⁻¹, amide I band 1645-1675 cm⁻¹, amide III at 1225-1300 cm⁻¹, and CH₃, CH₂ deformation of collagen at 1313, 1397, and 1440). Increased concentration of cholesterol esters (1739 cm⁻¹), carotenoids (1159 and 1523 cm⁻¹), calcifications (985 cm⁻¹), and hemoglobin (1250 and 1585 cm⁻¹) have also been reported. However, hemoglobin can be present in any excised tissue. GBM tissue has been shown to have lower lipid and cholesterol content than normal brain tissue, and higher nucleic acid content (1097 and 1580-1700 cm⁻¹). When compared with necrosis, tumor tissue should display increased lipids and nucleic acids. The Raman data from this study followed those trends. Normal grey matter showed strong contribution from lipid peaks, especially at 1061 and 1081 cm⁻¹, and necrosis had strong contributions from proteins, especially at 1003, 1206, 1239, 1255-1266, and 1552 cm⁻¹. Peaks associated with carotenoids (1154 and 1518 cm⁻¹), calcifications (977 cm⁻¹), and hemoglobin (1250 and 1581 cm⁻¹) were also elevated in necrosis. Necrosis also showed a broad shoulder at 1239 cm⁻¹. This region is associated with the amide III band of proteins. As conformation of the protein structure changes, the peak becomes broader, suggesting necrosis has a higher concentration of α-helix and random chain structures than normal and GBM tissues. GBM had a lower protein content than necrosis, as evidenced at 1003, 1030, 1206, 1239-1266, 1313, 1552, and 1657 cm⁻¹. The composition of 1061 and 1081 was lower in GBM than in normal grey matter, and higher than that of necrotic tissues. In the primary validation data set, overall accuracy was 97.8.

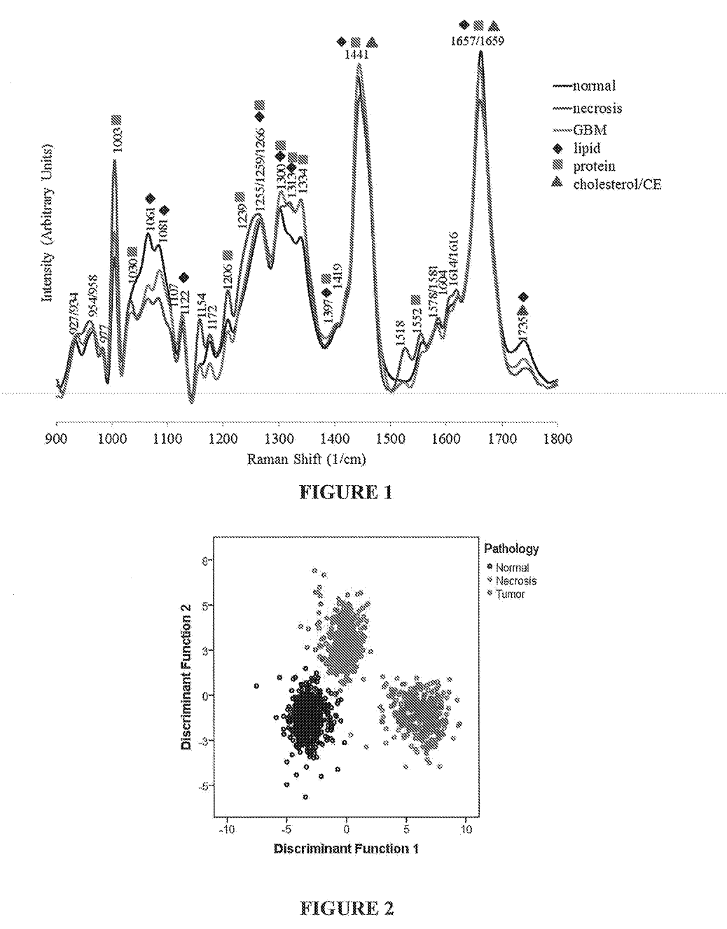

FIG. 2 shows a plot of the discriminant function analysis scores for data in the training set. Discriminant function 1 shows a distinct difference between normal and necrosis tissue, and a smaller distinction between normal and tumor tissue. Function 2 clearly separates tumor spectra from both normal grey matter and necrosis. The structure of discriminant function 1 showed significant peaks for distinguishing a continuous shift from normal to tumor to necrosis showed increasing protein content (phe at 1003, protein at 1155, amide III at 1239 and 1256, and nucleic acids at 1335 cm⁻¹) and decreasing lipid content (1062, and 1082 cm⁻¹) as tissue became necrotic. Interpretation of discriminant function 2 was more complex. Four peaks from the structure matrix described relationships that were applicable to both normal grey matter and necrosis. GBM had lower protein content than both normal and necrotic tissue, evidenced by the amide III backbone at 1552 cm⁻¹, nucleic acid peak at 1172 cm⁻¹, and phenylalanine at 1206 cm⁻¹. Conversely, GBM had a higher peak at 1122 cm⁻¹. Three other peaks seemed to describe the relationship just between GBM and normal grey matter. GBM had decreased lipid concentration from normal tissue at 1061 cm⁻¹, and increased nucleic acid and protein content at 1313 and 1334 cm⁻¹.

The 34 identified Raman peaks were split into combinations of peaks within 20 wavenumbers of each other. Discriminant function analysis was applied iteratively to each 20-wavenumber region to find the single region which provided the best overall classification accuracy between normal grey matter, necrosis, and GBM. The best region is shown as Region 1 in Table 1 below. Again, an iterative process was applied to each 20-wavenumber region to find the single region which added the next best classification accuracy to the training data when added to Region 1. This process was repeated until five key regions were identified. The specific peaks used are shown, as well as the increase in overall accuracy when each combination of peaks is added. The 34 original Raman peaks input to discriminant function analysis provided 99.6% accuracy, however, 98.5% overall accuracy can be achieved by using only five key regions. However, it is understood that any number of spectral regions can be combined that provide a desired combined classification accuracy.

TABLE 1

| | | Cumulative | | | | |
|---|---|---|---|---|---|---|
| | Peak(s) | Overall | Normal | Necrosis | Tumor | Raman |
| Region | added | accuracy | accuracy | accuracy | accuracy | assignment |
| 1 | 1657.6, | 78.9% | 84.6% | 74.8% | 72.9% | Amide I |
| | 1659.3 | | | | | |
| 2 | 1154.9, | 91.6% | 96.2% | 85.3% | 89.7% | Carotenoids (1154); |
| | 1172.6 | | | | | Unassigned (1172) |
| 3 | 1003.2 | 96.8% | 97.7% | 95.8% | 96.1% | Phenylalanine |
| 4 | 1107.0, | 97.7% | 98.5% | 96.9% | 97.2% | Nucleic acid (1107); |
| | 1122.1 | | | | | glycogen (1122) |
| 5 | 1255.8, | 98.5% | 98.9% | 98.3% | 97.9% | Amide III |
| | 1259.5, | | | | | |
| | 1266.8 | | | | | |

To confirm the accuracy of the model, it was applied to the primary and secondary validation sets. Results are shown below in Table 2 for each region measured. Overall accuracy in the primary validation group was 95.3%. Overall accuracy in the secondary training group was 71.3%. This group contained regions which displayed significant freeze artifact. The presence of freeze artifact may have altered the composition of the tissue, contributing toward the lower accuracy. Freeze artifact will not be present in in vivo tissues.

TABLE 2

| | | # | H&E | Raman DX | | |
|---|---|---|---|---|---|---|
| | Region Name | Pts | Diagnosis | #norm | #nec | #GBM |
| Training | hf156-093011-m1 | 260 | normal | 257 | 0 | 2 |
| Data | hf221-102711-m1 | 221 | normal | 218 | 0 | 2 |
| | hf572-110811-m2 | 176 | normal | 173 | 0 | 3 |
| | hf140a-092311-m1 | 160 | necrosis | 0 | 159 | 1 |
| | hf140a-092311-m2 | 130 | necrosis | 1 | 128 | 1 |
| | hfl220-113011-m3 | 63 | necrosis | 0 | 60 | 3 |
| | hf138-091911-m1 | 176 | GBM | 5 | 0 | 171 |
| | hf142s2-092911-m1 | 135 | GBM | 3 | 0 | 132 |
| | hf142-091911-m1 | 77 | GBM + MVP | 0 | 0 | 77 |
| Validation | hf221-102611-m1 | 221 | normal | 218 | 0 | 3 |
| Data | hf572-110811-m1 | 198 | normal | 198 | 0 | 0 |
| | hf572-110811-m3 | 165 | normal | 165 | 0 | 0 |
| | hf140a-092111-m1 | 153 | necrosis | 0 | 152 | 1 |
| | hf142s2-092911-m2 | 135 | GBM | 1 | 0 | 134 |
| | hf1220-113011-m1 | 140 | GBM | 49 | 5 | 82 |
| | hf1220-113011-m2 | 180 | GBM | 2 | 0 | 178 |
| | hf142-091911-m2 | 170 | GBM + MVP | 1 | 1 | 168 |
| Validation | hf140b-030612-m1 | 169 | normal + FA | 13 | 0 | 156 |
| Data (with | hf140b-030612-m2 | 132 | normal + FA | 9 | 0 | 123 |
| freeze | hf140b-091611-m2 | 136 | normal + FA | 66 | 0 | 65 |
| artifact) | hf151-092811-m1 | 345 | normal + FA | 310 | 0 | 34 |
| | hf201-101811-ml | 216 | normal + FA | 200 | 0 | 15 |
| | hf201-101811-m2 | 209 | normal + FA | 4 | 13 | 191 |
| | hf1178-120511-m1 | 180 | necrosis + FA | 2 | 175 | 3 |
| | hf212a-101911-m1 | 352 | necrosis + FA | 0 | 341 | 11 |
| | hf274-110111-m2 | 176 | necrosis + FA | 0 | 110 | 66 |
| | hf422-110911-m1 | 99 | necrosis + FA | 1 | 90 | 2 |
| | hf422-110911-m2 | 234 | necrosis + FA | 2 | 189 | 43 |
| | hf422-110911-m3 | 154 | necrosis + FA | 34 | 86 | 34 |
| | hf581-111111-m2 | 192 | necrosis + FA | 0 | 188 | 3 |
| | hf1255-112111-m2 | 204 | GBM + FA | 1 | 0 | 201 |
| | hf1255-112111-m3 | 144 | GBM + FA | 4 | 1 | 139 |
| | hf212a-101911-m2 | 204 | GBM + MVP + FA | 2 | 0 | 202 |
| | hf212b-101411-m1 | 260 | GBM + MVP + FA | 141 | 1 | 118 |
| | hf350-112311-m1 | 165 | GBM + MVP + FA | 31 | 14 | 120 |
| | hf350-112311-m2 | 238 | GBM + MVP + FA | 92 | 8 | 136 |
| | hf355-111511-m1 | 165 | GBM + MVP + FA | 15 | 2 | 147 |
| | hf355-111511-m2 | 192 | GBM + MVP + FA | 96 | 0 | 95 |
| | hf355-111511-m4 | 80 | GBM + MVP + FA | 2 | 2 | 76 |

Therefore, five Raman spectral regions have been identified as most significant for diagnosis and identification of normal grey matter, necrosis and GBM, including region 1 of 1657-1660, region 2 of 1153-1172, region 3 of 1002-1004, region 4 of 1106-1123, and region 5 of 1254-1268 wavenumbers. Based on these data, in one embodiment, the apparatus and method disclosed herein provides a spectral analysis of these five different, non-overlapping Raman spectral regions. It is understood that these specific spectral regions were selected for distinguishing between normal grey matter, necrosis, and GBM tumors in the brain, and therefore other spectral regions may be identified for distinguishing between other types of normal, necrotic, and tumor tissue. It is also understood that while five spectral regions are utilized herein, this number is not intended to be limiting, as more or fewer regions may be suitable for identifying alternative tissue types.

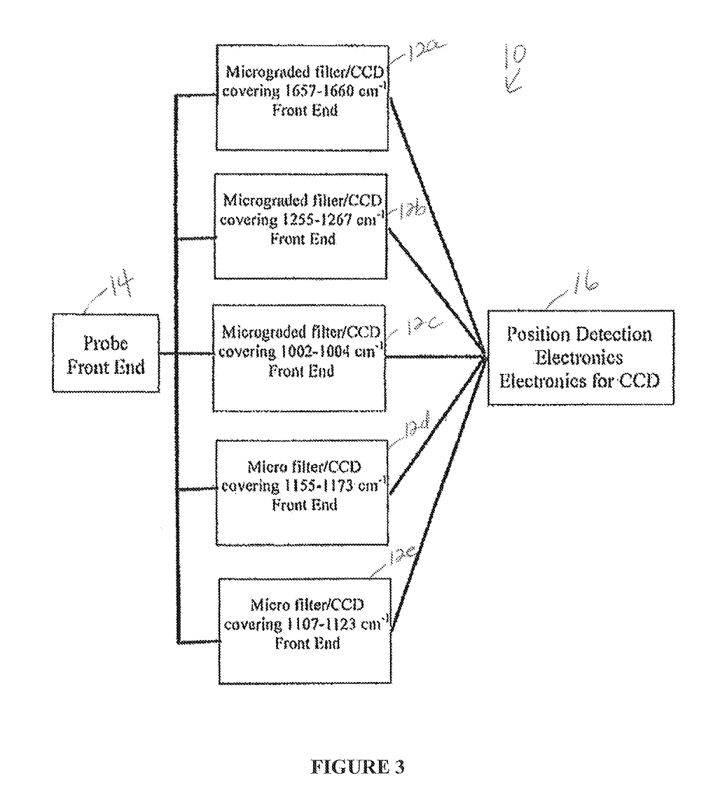

Embodiments of the apparatus and method disclosed herein use specific Raman regions, instead of a broad range of wavenumbers, to identify different tissue types. Any Raman peaks within a range of 20 cm⁻¹ can be measured by a single Raman chip/CCD detector or micro-Raman spectrometer, and an array of these spectrometers is combined in a single, portable apparatus 10 which is illustrated schematically in FIG. 3. The apparatus 10 includes a plurality of spectrometers 12a-12e, one for each of the five different spectral regions selected for distinguishing between normal grey matter, necrosis, and GBM. The apparatus 10 also includes a Raman probe 14 and a processor 16, including electronics for position detection and electronics for CCD. The processor 16 is in communication with the spectrometers 12, and analyzes output from the spectrometers 12 to identify the tissue type of a tissue sample. Such an apparatus 10 can be manufactured at a small, handheld size for operating room use, and at a lower cost, making the apparatus more accessible to all surgeons. In turn, processing will focus only on the data from the five small spectral regions covered, instead of data over a broad range of Raman wavenumbers. Again, it is understood that although five chips/spectrometers 12 and corresponding spectral regions are described herein, the disclosed apparatus 10 and method are not limited to this number nor the specific wavenumbers identified.

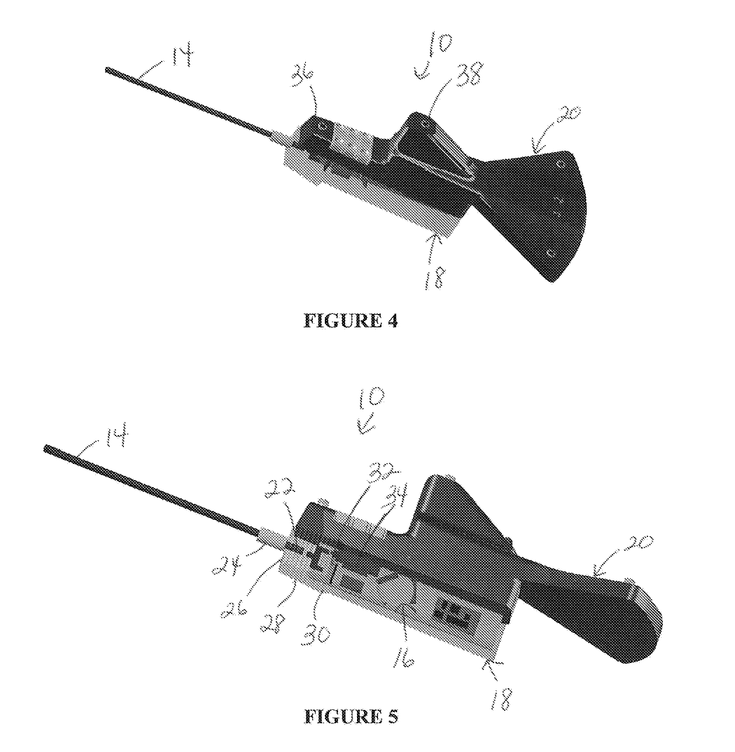

With reference to FIGS. 4 and 5, the apparatus 10 may comprise a housing 18 and further include an integrated tracking system 20 in communication with navigation software, such as to provide surgeons with the ability to detect and map the spatial coordinates of malignant regions of a tumor. The tracking system 20 provides surgeons with the ability to determine the position of the tip of the Raman probe 14, in real time, relative to an anatomical landmark of the patient during the operation. By including built-in tracking in the apparatus 10, surgeons are provided with the ability to detect as well as map the spatial coordinates of malignant regions, which is especially significant in identifying the type of tissue at the margins. Tissue movement may be compensated in real-time during the tracking by a machine vision system (not shown) that tags features of the surgical object and compensates for movements or shifts during the surgical procedure.

In addition to the tracking system 20, a light source (such as a laser diode) 22 for tissue illumination, excitation fibers 24, collection fibers 26, a collimating lens 28, an optical dispersing element 30, a CMOS detector 32, and a mirror 34 may be integrated into the handheld Raman apparatus 10. The wavelength of the light source 22 and other optical parameters may be defined based upon the optimal excitation wavelength and resultant Raman shifts described above. User controls 36 and a power indicator 38 may be provided, and the apparatus 10 may also be configured to provide a visible or audible indication of tissue type to the user.

In operation, the surgeon places the probe 14 adjacent the tissue and acquires the Raman spectra for the tissue to determine the tissue type. Light scattered by the tissue is collected by optics to be transmitted to the spectrometers (CCD) 12a-12e. The processor 16 analyzes spectra from the five spectrometers (CCD) 12a-12e, determining signal intensity in order to identify specific spectral characteristics of the Raman spectra received in real time to determine tissue type. The surgeon can also use the probe 14 to locate margins of the tumor by determining the points nearest to the tumor where the apparatus 10 indicates that the tissue is normal. Once the margins of the tumor have been identified, the entire tumor can be removed without removing excess tissue.

Although the processor 16 is shown as being contained within the housing, the processor may alternatively be a personal computer external to and in communication with the apparatus 10. The processor 16 can execute software instructions stored in a memory module (not shown) in communication with the processor 16 which causes the processor to perform the method disclosed herein. The software instructions may be stored on a computer-readable medium such as, but not limited to, physical media or electronic data storage media.

The disclosed apparatus and method can be used to identify tumor, necrosis, and normal tissue regions in vivo without damaging tissue. This is a need of every neurosurgeon who operates on brain cancer. The portability of the disclosed spectrometer apparatus and its design for Raman bands uniquely suited to neurosurgical applications make it ideal for in vivo tumor detection, mapping tissue boundaries, and glioblastoma resection surgery.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A portable apparatus for distinguishing between different tissue types in a tissue sample, the apparatus comprising:
a housing;
a plurality of Raman spectrometers disposed within the housing, each spectrometer having a different spectral region; and
a processor in communication with the plurality of spectrometers, the processor analyzing output from the plurality of spectrometers to identify the tissue type of the tissue sample.

2. The apparatus of claim 1, wherein the different tissue types include normal tissue, necrotic tissue, and tumor tissue.

3. The apparatus of claim 1, wherein the plurality of spectrometers includes five spectrometers.

4. The apparatus of claim 1, wherein each spectral region does not exceed 20 wavenumbers.

5. The apparatus of claim 1, wherein the spectrometers have a first spectral region including wavenumbers 1657-1660, a second spectral region including wavenumbers 1153-1172, a third spectral region including wavenumbers 1002-1004, a fourth spectral region including wavenumbers 1106-1123, and a fifth spectral region including wavenumbers 1254-1268.

6. The apparatus of claim 1, wherein the different tissue types include normal grey matter brain tissue, necrotic brain tissue, and glioblastoma tumor tissue.

7. The apparatus of claim 6, wherein the processor identifies a Raman spectrum for necrotic brain tissue as being characterized by an increased protein content and a decreased lipid content compared to normal brain tissue.

8. The apparatus of claim 6, wherein the processor identifies a Raman spectrum for glioblastoma as being characterized by a decreased lipid content, a decreased cholesterol content, and an increased nucleic acid content compared to normal grey matter brain tissue.

9. The apparatus of claim 6, where the processor identifies a Raman spectrum for glioblastoma as being characterized by an increased lipid content, and increased nucleic acid content, and a decreased protein content compared to necrotic brain tissue.

10. The apparatus of claim 1, further comprising a probe connected to the housing, and a light source disposed within the housing for illuminating the tissue sample via the probe.

11. The apparatus of claim 10, wherein the apparatus further includes a tracking system to detect a position of the probe in real time relative to an anatomical landmark associated with the tissue sample.

12. A method for distinguishing between different tissue types in a tissue sample, the method comprising:
providing a portable apparatus having a housing, a light source disposed within the housing, and a plurality of Raman spectrometers disposed within the housing, each spectrometer having a different spectral region;
illuminating the tissue sample using the light source;
receiving light from the tissue sample with the plurality of spectrometers; and
analyzing output from the plurality of spectrometers to identify the tissue type of the tissue sample.

13. The method of claim 12, wherein the different tissue types include normal tissue, necrotic tissue, and tumor tissue.

14. The method of claim 12, wherein the different tissue types include normal grey matter brain tissue, necrotic brain tissue, and glioblastoma tumor tissue.

15. The method of claim 12, wherein the plurality of spectrometers includes five spectrometers.

16. The method of claim 12, wherein each spectral region does not exceed 20 wavenumbers.

17. The method of claim 12, wherein spectrometers have a first spectral region including wavenumbers 1657-1660, a second spectral region including wavenumbers 1153-1172, a third spectral region including wavenumbers 1002-1004, a fourth spectral region including wavenumbers 1106-1123, and a fifth spectral region including wavenumbers 1254-1268.

18. The method of claim 12, further comprising identifying boundaries between the different tissue types.

19. The method of claim 12, further comprising detecting a position of the apparatus in real time relative to an anatomical landmark associated with the tissue sample.

20. A method for distinguishing between different tissue types in a tissue sample using different Raman spectral regions, the method comprising:
(a) selecting a first spectral region which provides a best classification accuracy between the tissue types;
(b) selecting a next spectral region that provides a next best classification accuracy between the tissue types;
(c) repeating step (b) until a plurality of spectral regions are selected that, when combined, provide a desired combined classification accuracy; and
(d) analyzing the tissue sample with the plurality of selected spectral regions to determine the tissue type in the tissue sample.
